(19) United States
(12) Patent Application Publication
(10) Pub. No.: US 2011/0022037 A1
(43) Pub. Date: Jan. 27, 2011

(54) System and Method for Minimizing the Side Effects of Refractive Corrections Using Line or Dot Cuts for Incisions

(76) Inventors: Josef F. Bille, Heidelberg (DE); Kristian Hohla, Munich (DE); Frieder Loesel, Mannheim (DE)

Correspondence Address: NYDEGGER & ASSOCIATES, 348 OLIVE STREET, SAN DIEGO, CA, 92103, US

(21) Appl. No.: 12/868,608

(22) Filed: Aug. 25, 2010

Related U.S. Application Data
(63) Continuation-in-part of application No. 12/349,257, filed on Jan. 6, 2009.

(51) Int. Cl. A61F 9/008

(52) U.S. Cl. 606/5

(57) ABSTRACT

A system and method for performing refractive surgery in an eye requires creating a plurality of cuts in the stroma or the lens that are randomly positioned relative to a reference axis. The geometry for each cut is unique and includes a start point in the stroma that is identified by a distance “r” from the axis, and an azimuthal angle “θ” that is measured around the axis. A computer provides concerted control for a laser unit and an optical scanner to randomly vary the start point for each cut, to create a pattern of cuts that will implement the desired refractive surgery, yet be visually illusive.

This application is a continuation-in-part of application Ser. No. 12/349,257 filed Jan. 6, 2009, which is currently pending. The contents of application Ser. No. 12/349,257 are incorporated herein by reference.

### FIELD OF THE INVENTION

The present invention pertains generally to systems and methods that are useful for altering the refractive properties of a transparent material. More specifically, the present invention pertains to systems and methods that weaken tissue on an eye with a laser beam, to correct vision defects of the eye. The present invention is particularly, but not exclusively, useful as a system and method for weakening eye tissue with laser incisions constituting linear or dot cuts that are statistically distributed (i.e., pseudo-random), to thereby become visually elusive for minimizing adverse visual side effects that might otherwise be introduced.

### BACKGROUND OF THE INVENTION

PresbyLASIK is an excimer laser based method that is used to achieve a multifocal cornea that restores near vision in presbyopic patients. PresbyLASIK is also sometimes called multifocal LASIK because it works on principles virtually identical to the artificial multifocal lenses that provide vision correction for presbyopes, especially presbyopic cataract patients. Essentially, there are two types of presbyLASIK procedures. One is central presbyLASIK wherein a central disk is created for near vision and a peripheral ring is established for distance vision. The other is peripheral presbyLASIK wherein a central disk is created for distance vision and a mid-peripheral ring is created for near vision. It has been shown that the visual results of both types of presbyLASIK procedures compare favorably to the best pseudo-accommodation that could be theoretically achieved using multifocal refractive intraocular lenses. In these LASIK cases, however, there is still the possibility of introducing uncertainties that may compromise the final refractive outcome.

A recently presented procedure for introducing refractive corrections (e.g. the correction of presbyopia) into the cornea of an eye, without LASIK, involves the weakening of tissue in the stroma. Specifically, such a procedure is disclosed in U.S. Pat. No. 7,717,907 which issued on May 18, 2010 for an invention entitled “Method for Intrastromal Refractive Surgery,” (hereinafter the '907 patent) which is assigned to the same assignee as the present invention. As disclosed in the '907 patent, the weakening of stromal tissue is accomplished using a pulsed femtosecond laser beam to create incisions in the stroma. It can happen in a small number of cases, however, that these incisions may introduce annoying side-effects in night-vision (e.g. halos, ring-patterns around bright light sources). Further, annoying side-effects may result inherently due to the multifocality of the reshaped cornea. Apart from refractive corrections in the stroma of an eye, it is also known that vision corrections sometimes require surgery on the lens of an eye. More specifically, similar to stromal cuts, certain visual corrections for the lens can be made by making cuts into the lens tissue. Like refractive corrections for the stroma, however, cuts into the lens of an eye may also introduce annoying side effects. In any event, for either the stroma or the lens, the unwanted side-effects are preferably overcome via neuro-adaptive suppression.

In one of its aspects, neuro-adaptive suppression involves having the brain effectively ignore a visual perception. For example, it can be demonstrated that a plethora of irregularities (e.g. stromal incisions) may be presented in a manner that will visually disguise an underlying regularity. Insofar as an ophthalmic laser procedure is concerned, such a neuro-adaptive suppression may be very advantageous. In particular, this will be so if the collective irregularities simultaneously accomplish a two-fold purpose. For one, a pattern of collective irregularities must accomplish the same refractive correction that would otherwise be obtained by the underlying regularity alone. For another, the pattern of irregularities needs to be visually illusive (i.e. obfuscate the underlying regularity), and thereby minimize any annoying visual side-effects (e.g. halos) that might otherwise arise.

In light of the above, it is an object of the present invention to provide a system and method for minimizing visual side-effects of a refractive surgical procedure that achieves a desired refractive correction with a pattern of incisions constituting linear or dot cuts. Another object of the present invention is to provide a system and method for minimizing visual side-effects of a refractive surgical procedure that achieves a desired refractive correction with a random or pseudo-random pattern of cuts. Another object of the present invention is to provide such a random or pseudo-random pattern of cuts, based on a statistical distribution. Still another object of the present invention is to provide a system and method for minimizing visual side-effects of a refractive surgical procedure that is simple to use, relatively easy to manufacture, and comparatively cost effective.

### SUMMARY OF THE INVENTION

In accordance with the present invention, a system for performing laser refractive surgery on either the stroma or lens of an eye includes a laser unit, an optical scanner unit and a computer. In combination, the laser unit and the optical scanner unit are computer-controlled to create a pattern of straight line cuts inside the tissue (lens or stroma) of the eye. The purpose here is two-fold. For one, the cuts are intended to weaken biomechanical stress distributions in the tissue in a manner that provides for a desired refractive vision correction. For another, the cuts are statistically distributed in the pattern to become visually illusive.

In detail, the laser unit is used for generating a pulsed laser beam. Preferably, each pulse of the laser beam will be less than one picosecond in duration, and each pulse will have an energy level that is less than about 20 μJ. As is well known, pulses with such operational parameters are capable of causing a Laser Induced Optical Breakdown (LIOB) of tissue. In order to employ this capability, the optical scanner unit is connected with the laser unit for the purpose of moving the focal spot of the laser beam through the stroma or lens, to perform LIOB at successive focal spots in the tissue of the eye. Specifically, this is done to create a plurality of cuts in the tissue (i.e. a pattern) that will accomplish the two-fold purpose of the present invention.

As indicated above, the computer is electronically connected to both the optical scanner unit and to the laser unit. Within this connection, movements of the laser beam focal spot are determined by the optical scanner unit. Also, these movements can be coordinated with the generation of the pulsed laser beam by the laser unit. All of this is done in accordance with a computer program.

In essence, the computer program for the present invention defines a plurality of cuts that are collectively used to establish a pattern for the cuts. In detail, the computer defines each cut as having three orthogonal dimensions (e.g. x-y-z). Further, depending on the type and shape of the particular cut that is to be made, any one of these dimensions can be varied. Also, each cut may be less than about ten microns in length. In this last case, where all three dimensions (x-y-z) are less than ten microns, the cut is essentially a “dot.” On the other hand, when any one (but only one) of the three dimensions is extended beyond ten microns, the cut then effectively becomes a “line.” Further, because a “line” cut is essentially a sequence of “dot” cuts, a “line” cut can be made to be either straight or curved. When a plurality of cuts are made (“dots” or “lines”), the result is the pattern of cuts mentioned above. As envisioned for the present invention, such a pattern may constitute only “dots,” only “lines,” or a combination of “dots” and “lines.”

Insofar as so-called “line” cuts are concerned, they will typically lie along substantially straight paths that are defined by the computer program. For purposes of disclosure, these straight paths (line cuts) can be described relative to a reference axis. Preferably, the axis can be defined by the eye and will be either a visual axis, an optical axis, a line-of-sight axis, a pupillary axis or a compromise axis. According to the program, each path will have a particularly unique orientation in the stroma, and each path will extend between an intersection point on the reference axis and a set point on the anterior surface of the eye. Further, each path may, but not necessarily, be perpendicular to the anterior surface of the eye, and will follow a respective straight line inside the stroma. As envisioned for the present invention, the intersection point on the reference axis may be either anterior or posterior to the anterior surface of the eye. Typically, the length of each line cut is less than approximately 400 microns with a separation distance between adjacent line cuts greater than about 5 microns in most instances. Further, the diameter of each line cut, from end-to-end of the line cut, will generally be less than about 3 microns.

In an implementation of the system of the present invention, each cut in a pattern has a start point that is located at a distance “r” from the axis. The start point is located inside the stroma material of the eye, or the lens of the eye, at an azimuthal angle “θ” that is measured in a plane perpendicular to the axis. Both the distance “r” and the azimuthal angle “θ” are unique for each cut. In the case of a line cut, the line cut begins at a unique start point on a particular path and extends therefrom toward the reference axis through a distance “d” in the stroma. Importantly, within this geometry, each line cut is oriented to intersect the reference axis at an inclination angle “φ”. With the start point at a distance “l” from the reference axis, as measured along the path, the inclination angle can be defined as φ=arcsin r/l.

In addition to components of the system disclosed, the system may also include a stabilizing device that can be connected to the optical scanner unit in any manner known in the pertinent art. If used, the stabilizing device holds the axis of the eye substantially stationary during the surgery. Also, the computer program can be used to define a thickness and a topography of the anterior surface of the eye and to define multiple cuts into the stroma in accordance with the thickness or the topography. Importantly, for all embodiments of the present invention, the computer program randomly establishes a plurality of start points. And, the computer program may also be used to randomly establish the location of each “dot” cut and the lengths of each “line” cut. All of this is done in order to create the visual illusiveness of the pattern.

From another perspective, the present invention can be considered as being a compilation of three interactive computer programs. In this perspective, there is a first program for defining each cut. Specifically, the first program defines the reference axis and, in line with the disclosure above, it also establishes a start point for each cut inside the stroma or the lens. As disclosed above, each start point is located at a distance “r” from the reference axis. Further, the start point is located at an azimuthal angle “θ” that is measured in a plane perpendicular to the axis. And, in the case of line cuts, the line cut extends from the start point through a distance “d” along the path toward the reference axis. As indicated above, “r” and “θ” for each “dot” cut is unique, and “r”, “θ”, “φ” and “d” for each line cut is unique. Once the orientation of a line cut has been established (i.e. the first program), the second program establishes a pattern having a plurality of dots and/or line cuts. In the second program, although each cut is defined, it has a unique start point and “r” and “θ” are randomly selected, as required, in order to achieve the desired visual illusiveness of the refractive correction. With the third computer program, the optical scanner controls the laser beam to create the pattern of cuts in the stroma or lens.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the system components of the present invention shown positioned for creating cuts in the stroma or lens of an eye in accordance with the present invention;

FIG. 2 is a cross section view of line cuts located inside the tissue of an eye, shown relative to the visual axis of the eye;

FIG. 3A is a top plan view of a cornea of an eye;

FIG. 3B is a cross section view of the cornea as seen along the line 3B-3B in FIG. 3A showing, for purposes of disclosure, respective patterns of line cuts and dot cuts; and

FIG. 4 is a cross section view of the lens of an eye.

### DESCRIPTION OF THE PREFERRED EMBODIMENTS

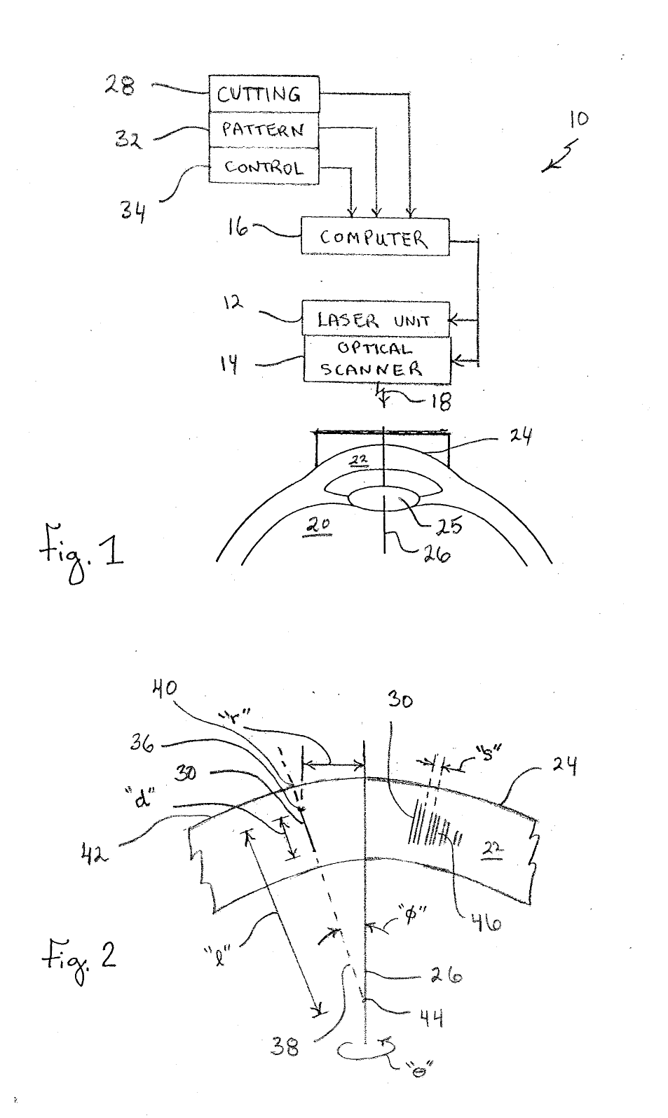

Referring initially to FIG. 1, a system for performing refractive surgery on an eye (transparent material) is shown and is generally designated 10. As shown, the system 10 essentially includes a laser unit 12, an optical scanner 14 and a computer 16. In combination, the computer 16 is used to control both the laser unit 12 and the optical scanner 14 to respectively generate and direct a pulsed laser beam 18. The laser beam 18 is then used to perform refractive surgery on an eye 20. More specifically, the system 10 directs and focuses the laser beam 18 into the stromal tissue 22 of the cornea 24 of an eye 20 or into the lens 25 of the eye 20 to perform this surgery.

For purposes of control, the laser beam 18 is moved relative to a reference axis 26 that is defined by the eye 20. This reference axis 26 can be either a visual axis, an optical axis, a line-of-sight axis, a pupillary axis or a compromise axis. For operational purposes, the laser beam 18 can have any characteristics well known in the pertinent art that are capable of causing Laser Induced Optical Breakdown (LIOB) on the stromal tissue 22 (e.g. a so-called femto-second laser). Preferably, pulses of the laser beam 18 will have a less than one picosecond duration, with an energy level for each pulse of less than about twenty microjoules (20 μJ). Thus, the spot size for LIOB will be approximately five microns (5 μm).

Still referring to FIG. 1, it is to be appreciated that an operation of the computer 16 essentially involves three definable, but interrelated, input programs. Although these programs will operate together in concert with each other, for disclosure purposes they can be considered separately. Functionally, these programs include: 1) a cutting program 28 for individually defining each cut 30 (30′) inside the stromal tissue 22 (see FIG. 2); 2) a pattern program 32 for generating a pattern that is made by a plurality of the cuts 30 (30′); and 3) a control program 34 for moving the laser beam 18 to create each cut 30 (30′) by LIOB. Note: for purposes of disclosure, the cuts 30 are created as a line (i.e. line cut 30) and the cuts 30′ are created as dots (i.e. dot cuts 30′).

Referring now to FIG. 2, a line cut 30 in accordance with the present invention is shown to begin at a start point 36 inside the stromal tissue 22. As shown, the line cut 30 is straight and it lies on a path 38 (dashed line). Further, the path 38 extends from a set point 40 on the anterior surface 42 of the cornea 24, to an intersection point 44 on the reference axis 26. For the present invention, the intersection point 44 can be either anterior or posterior to the anterior surface 42 of the cornea 24.

FIG. 2 also shows that the start point 36 for each line cut 30 is established at a respective distance “r” from the reference axis 26, and that it (i.e. line cut 30) has a length “d” on the path 38. Also, the start point 36 is established inside the stromal tissue 22 and located at an azimuthal angle “θ” that is measured in a plane perpendicular to the reference axis 26. Also, the path 38 (i.e. the line cut 30) is tilted relative to reference axis 26 at an inclination angle “φ”. Thus, with the start point 36 located at a distance “l” from the intersection point 44 (i.e. “l” is measured from start point 36 along the path 38 toward the intersection point 44), the inclination angle “φ” can be defined as φ=arcsin r/l.

With the parameters identified above, each line cut 30 is created separately, and the individual parameters “d”, “r”, “θ”, and “φ” are unique for each particular line cut 30. Importantly, each line cut 30 is created by the laser beam 18 only inside the stromal tissue 22. Dimensionally, a line cut 30 will be created that is less than approximately 400 microns in its length (i.e. d<400 μm), and has a diameter that is less than about 5 microns. Typically, the separation distance “s” between adjacent line cuts 30 will be in a range between 5 and 10 microns (5 μm<s<10 μm).

As indicated above, in addition to the creation of line cuts 30, the present invention also envisions the creation of dot cuts 30′. In particular, each dot cut 30′ can be considered as being a small sphere and, accordingly, it is three dimensional. Specifically, it can be defined by three orthogonal dimensions that are measured in an arbitrarily established reference system (e.g. x-y-z). With this in mind, when LIOB is performed with all three dimensions (i.e. x-y-z) being less than about ten microns, the result is a dot cut 30′. In this case, with reference to the axis 26, the identifying parameters for a dot cut 30′ are “r” and “θ”. When one, but only one, of the dimensions for a dot cut 30′ is extended substantially beyond ten microns, as when LIOB is performed at successively connected focal spots, the result is a line cut 30. As noted above, for a line cut 30 the identifying parameters are again, “d”, “r”, “θ” and “φ”.

Once the line cuts 30 (dot cuts 30′) have been defined by the cutting program 28, a plurality of them (line cuts 30 and/or dot cuts 30′) are arranged in a pattern 46 to accomplish the desired refractive surgery. It is an important aspect of the system 10, however, that this pattern 46 be randomly generated by the pattern program 32. Recall, the parameters for each line cut 30 (dot cut 30′) are all unique. Specifically, this is done so the arrangement of cuts 30/30′ becomes visually illusive. Nevertheless, despite the randomness of cuts 30/30′ within the pattern 46, there are general boundaries within the stromal tissue 22 that, as a practical matter, need to be heeded.

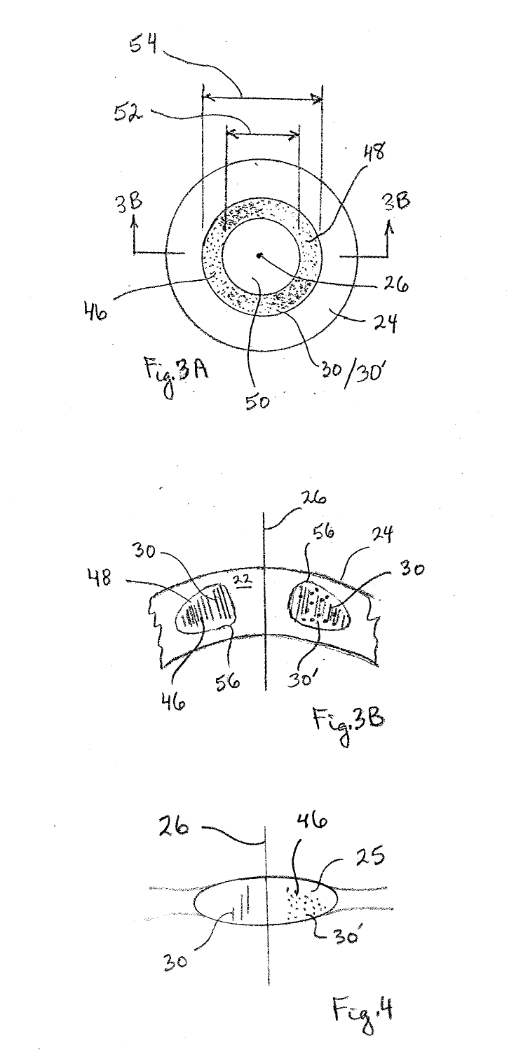

As will be appreciated by the skilled artisan, for intrastromal surgery, line cuts 30 and dot cuts 30′ will typically be confined in an annular shaped volume 48 that is located inside the stromal tissue 22. In each instance, this annular shaped volume 48 will surround a free central zone 50, and it will be centered on the reference axis 26. FIG. 3A shows that an inner diameter 52 effectively defines the free central zone 50. The annular volume 48 then extends outwardly from the inner diameter 52 to an outer diameter 54. Further, FIG. 3B shows that, between the diameters 52 and 54, the annular volume 48 is defined by a boundary 56. It is within this boundary 56 that LIOB is performed for the creation of the line cuts 30 or dot cuts 30′. Depending on the particular refractive correction that is to be made, the dimensions of the boundary 56 can be varied. Also, the density of line cuts 30 or dot cuts 30′ within the annular volume 48 can be varied.

As will be further appreciated by the skilled artisan, surgery on the lens 25 of an eye 20 can be accomplished in procedures similar to those disclosed above for intrastromal surgery. The only real limitation here involves the anatomy of the lens 25. In particular, care must be taken with regard to the interface between the lens 25 and its connective tissue (e.g. the capsule of the lens 25). With this in mind, and with reference to FIG. 4, it will be seen that a pattern 46 of cuts 30/30′ can be effectively made throughout the lens 25.

While the particular System and Method for Minimizing the Side Effects of Refractive Corrections Using Line or Dot Cuts for Incisions as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing laser refractive surgery on an eye, wherein the eye defines a reference axis and the system comprises:
a laser unit for generating a pulsed laser beam to provide for the Laser Induced Optical Breakdown (LIOB) of tissue at successive focal spots in the tissue of the eye;
an optical scanner unit connected with the laser unit for moving the focal spot of the laser beam through the tissue to create a plurality of cuts at respective focal spots, wherein each cut is defined by three mutually orthogonal dimensions and at least two of the dimensions of the cut are less than ten microns in length; and
a computer electronically connected with the laser unit and with the optical scanner unit for controlling the generation of pulsed laser beams by the laser unit and for controlling the movement of the focal spots of the laser beams in accordance with a computer program.

2. A system as recited in claim 1 wherein the pulses of the laser beam have a less than one picosecond duration with an energy level for each pulse less than 20 μJ.

3. A system as recited in claim 1 wherein the axis is selected from a group including a visual axis, an optical axis, a line-of-sight axis, a pupillary axis and a compromise axis.

4. A system as recited in claim 1 further comprising a stabilizing device connected to the optical scanner unit for stabilizing the eye, to hold the axis of the eye substantially stationary during the surgery.

5. A system as recited in claim 1 wherein all three orthogonal dimensions of the cut are less than ten microns.

6. A system as recited in claim 1 wherein the computer program defines a plurality of straight paths, wherein each path is unique and extends along a line between an intersection point on the reference axis and a set point on a surface of the eye, and further wherein each cut lies on a portion of a respective path inside the stroma.

7. A system as recited in claim 6 wherein each cut has a start point inside the stroma material of the eye at a distance “r” from the axis, with the start point located at an azimuthal angle “θ” measured in a plane perpendicular to the axis, and wherein the cut extends from the start point through a distance “d” in the stroma, and each cut is oriented to follow a straight line intersecting the axis at an inclination angle “φ)”, and wherein the start point is at a distance “l” from the axis in a direction along the line cut with φ=arcsin r/l.

8. A system as recited in claim 1 wherein the computer program further defines a thickness of the cornea of the eye and defines multiple cuts into the stroma in accordance with the thickness.

9. A system as recited in claim 1 wherein each cut has a start point and the computer program randomly establishes a plurality of start points.

10. A system as recited in claim 1 wherein the computer program creates a plurality of cuts within a defined volume inside the tissue of the eye.

11. A computer system for controlling an optical scanner of a laser unit, wherein the laser unit generates a laser beam and the computer system comprises:
a first program for defining a cut relative to a reference axis, wherein the reference axis has a fixed relationship with a surface of a transparent flexible material, wherein the cut has a unique start point inside the material and wherein each cut is defined by three mutually orthogonal dimensions and at least two of the dimensions of the cut are less than ten microns in length;
a second program, responsive to the first program, for creating a pattern having a plurality of randomly established cuts; and
a third program for controlling movement of the laser beam with the optical scanner, wherein the third program is electronically connected with the optical scanner for moving a focal spot of the laser beam in accordance with the pattern of cuts to perform Laser Induced Optical Breakdown (LIOB) of the transparent material.

12. A computer system as recited in claim 11 wherein the start point for each cut is at a distance “r” from the axis, and is located at an azimuthal angle “θ” measured in a plane perpendicular to the axis, and further wherein the cut extends from the start point through a distance “d” in the material.

13. A computer system as recited in claim 11 wherein a plurality of cuts are established within a defined volume inside the material in accordance with the second program, and wherein all three orthogonal dimensions of the cut are less than ten microns.

14. A computer system as recited in claim 11 wherein the surface of the transparent material is curved, wherein each cut is established on a linear path in the material and is oriented to intersect the axis at an inclination angle “φ”, and wherein the start point is at a distance “l” from the axis in a direction along the path.

15. A computer system as recited in claim 14 wherein the start point of the cut is at a distance “r” from the axis and φ=arcsin r/l.

16. A computer system as recited in claim 14 wherein the transparent flexible material is selected from a group comprising a cornea of an eye and a lens of an eye.

17. A method for controlling an optical scanner of a laser unit, wherein the laser unit generates a laser beam, the method comprising the steps of:
defining a reference axis having a fixed relationship with a surface of a transparent flexible material;
defining a plurality of start points relative to the axis inside the material, wherein each start point is at a unique distance “r” from the axis, and each start point is located at a unique azimuthal angle “θ” measured in a plane perpendicular to the axis;
creating a pattern having a plurality of cuts, wherein each cut is defined by three mutually orthogonal dimensions and at least two of the dimensions of the cut are less than ten microns in length, and further wherein each cut has a start point and the plurality of start points is randomly established for the pattern;
generating a pulsed laser beam; and
moving a focal point of the laser beam along the pattern of start points to perform Laser Induced Optical Breakdown (LIOB) of the transparent material to make a plurality of cuts into the material.

18. A method as recited in claim 17 wherein all three orthogonal dimensions of the cut are less than ten microns.

19. A method as recited in claim 17 further comprising the step of establishing the plurality of cuts within a defined volume inside the material, wherein each cut is a line cut and extends from a respective start point through a distance “d” in the material.

20. A method as recited in claim 19, wherein the surface of the transparent material is curved, and each cut is oriented to intersect the axis at an inclination angle “φ”, and wherein the start point is at a distance “l” from the axis in a direction along the line cut (φ=arcsin r/l).
